(19) United States
(12) Patent Application Publication
(10) Pub. No.: US 2011/0201901 A1
(43) Pub. Date: Aug. 18, 2011

(54) Systems and Methods for Predicting Patient Health Problems and Providing Timely Intervention

(76) Inventor: Sukhwant Singh Khanuja, Chicago, IL (US)

(21) Appl. No.: 13/029,474

(22) Filed: Feb. 17, 2011

Related U.S. Application Data
(60) Provisional application No. 61/305,259, filed on Feb. 17, 2010.

(51) Int. Cl. A61B 5/00

(52) U.S. Cl. 600/300

(57) ABSTRACT

A system and method is provided which predicts patient health problems so that timely help may be provided to the patient. In one embodiment, a patent monitors one or more of their biometric characteristics using a biometric data reader for at least several days. The biometric data is then passed to a central server that develops a model of the patient's normal biometric readings and normal procedures for taking a biometric reading including time of day and frequency of readings. Later readings are compared to the patient's model and a significant deviation from the model by the patient is correlated with patient data like diagnosis, claims history, demographics, etc to predict the onset of a health problem.

### CROSS-REFERENCE TO RELATED APPLICATIONS

The present application claims the benefit of U.S. Provisional Application No. 61/305,259, filed Feb. 17, 2010 entitled “Method For Predicting Patient Health And For Providing Personalized Care And Triggering Timely Intervention.”

### BACKGROUND OF THE INVENTION

The present invention generally relates to providing patients with timely health care for their health problems. More specifically, the present invention relates to providing timely health care by predicting patient health problems.

Patients fall sick and are treated for their specific identified ailments. This action is reactive and does not prevent the patient from decompensating or their health deteriorating, and possibly ending up in an emergency room and/or being hospitalized. Usually active treatment only starts once the patient is in clinical care. Timely intervention could prevent patient decompensation and thus clinical healthcare treatment including an emergency room visit or hospitalization. Preventing these clinical treatments would save a lot of money for the payer for these treatments as well as improve the quality of patients' lives.

Many people have tried to improve patient health and thereby avoid clinical healthcare treatments over time. These attempts have primarily used demographic data (age, sex, ethnicity etc,) along with Health Risk Assessment (HRA) which could include genetics, current diseases etc. However, pharmaceutical refill data has a built in delay—and lab tests need to be ordered and take time to arrive. Therefore, most prediction of a person's health is statistical suggesting for example someone may have a 60% risk of decompensating in 10 days after a surgery. However, these predictions are not based on personalized data and do not a have a good specificity.

### BRIEF SUMMARY OF THE INVENTION

One or more of the embodiments of the present invention provide systems and methods for predicting patient health problems so that timely help may be provided to the patient. In one embodiment, a patent monitors one or more of their biometric characteristics using a biometric data reader for at least several days. The biometric data is then passed to a central server that develops a model of the patient's normal biometric readings and normal procedures for taking a biometric reading including time of day and frequency of readings along with their disease state, claims history and demographic information. Later readings are compared to the patient's model and a significant deviation from the model by the patient is correlated to the onset of a health problem.

### BRIEF DESCRIPTION OF THE DRAWINGS

FIG. 1 illustrates a patient health intervention system according to an embodiment of the present invention.

FIG. 2 illustrates a flowchart of the patient health intervention system.

FIG. 3 illustrates an example of the prediction of the onset of a health problem.

### DETAILED DESCRIPTION OF THE INVENTION

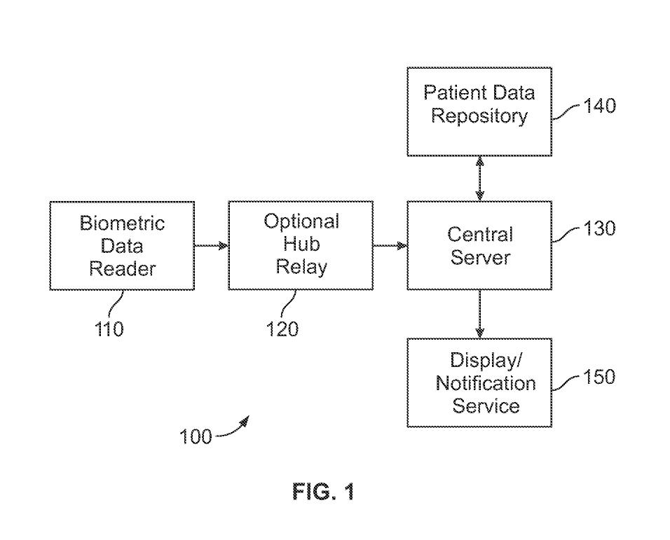

FIG. 1 illustrates a patient health intervention system 100 according to an embodiment of the present invention. The patient health intervention system 100 includes a biometric data reader 110, an optional hub relay 120, a central server 130, a patient data repository 140, and a notification/display service 150.

In operation, biometric data is read from a patient at the biometric data reader 110 and then passed to the central server 130. The biometric data may pass through the optional hub relay 120 if the hub relay 120 is present. Once data is received at the central server 130, it may be stored in the patient data repository 140, along with other data elements in the data repository like patients' diagnosis, claims history, demographic information. Patient data may later be retrieved and displayed from the patient data repository 140 using a display/notification service 150, which may for example be a computer application operating over a network or the internet.

In a typical situation, a patient may use the biometric data reader 110 to periodically monitor one of more of their biometric characteristics, such as blood pressure, glucose level, or weight. The patient has typically been performing the measurements of their biometric characteristics on a periodic basis for some time. For example, a patient may have been instructed by their doctor to take blood pressure readings twice a day. These readings are then relayed to the central server 130, typically for storage in the patient data repository, so that the records of the readings may be reviewed by a doctor or other caregiver if desired.

Because the patient has been periodically taking readings over several days, the readings may be synthesized or processed to determine a “normal” condition for the patient. Further, a substantial deviation from the “normal” condition may be determined to signal the likely onset of a health problem.

More specifically, a “normal” condition may be determined for several parameters including: 1) time of day at which the measurements are being taken, 2) the number of measurements taken in a day, 3) the time between individual measurements, and 4) the actual values of the measurements. It has been determined that patient deviation from “normal” patterns of measurement is correlated with an increase likelihood that the patient will soon experience the onset of a health problem—often requiring hospitalization. Further, more than one of the above parameters may be statistically combined as further described below.

For example, with regard to the time of day at which measurements are typically taken by the patient, consider the situation wherein the patient has been on a regimen of measuring their blood pressure twice a day and has typically taken one reading between 7 am and 8 am and the other between 4 pm and 5 pm over a period of a month. Suddenly, the patient takes a reading at 3 am in the morning. In this situation, the patient has departed from the normal time of day at which measurements are taken and the deviation may indicate the onset of a health problem.

Similarly, if the patient has typically been taking two readings/day, but starts taking three or four readings in a day, the deviation may indicate the onset of a health problem.

Also, in the example above, if the patient typically takes a single reading between 7 am and 8 am, but starts taking two or more readings during that time, the deviation may indicate the onset of a health problem.

Finally, the actual values of the biometric readings may indicate the onset of a health problem. Further, although some prior art systems may use basic, general population values to indicate the onset of a health problem, one or more embodiments of the present invention individual tailors the analysis of the actual values of the biometric readings to provide a personalized, and more accurate prediction of the onset of a health problem.

For example, prior art systems may sound an alert whenever any patient's systolic blood pressure passes a certain threshold, such as 120, for example. Conversely, the present system may monitor a patient who has developed a “normal” condition of systolic blood pressure of 150 and may then sound an alert when the patient's blood pressure exceeds 150 for a significant time. For example, if the patient's blood pressure exceeds 150 on both of the patient's next two readings which are, for example, 3 hours apart.

In addition to the determination of “normal” described above, the present system recognizes and incorporates weekends, and their potential deviation from the other days of the week, into its model. Since patients may have different daily routines on different weekdays, “normal” is calculated for a day for the week and deviation is measured from the “normal” for that weekday.

For example, patients may take reading between 6 am to Sam on weekdays and Sam to 10 am on weekends. Similarly, their normal readings values may be different on each day of the week.

Additionally, with regard to time-based readings, readings that depart from a patient's typically schedule and take place during the night when the patient has been sleeping have been found to have an increased correlation with the onset of a health problem. For example, if a patient is not only deviating from the typical time at which they take their blood pressure reading, but is actually taking the reading at 3 am in the morning, there is an additional likelihood of the onset of a health problem.

Another example, of correlating multiple readings, is when a patients takes weight readings at for example 2 am in the night and then in the morning reports a peak flow (PEF/FEV1) readings which are lower than normal. This may indicate they did not sleep well and also may indicate the onset of a health problem.

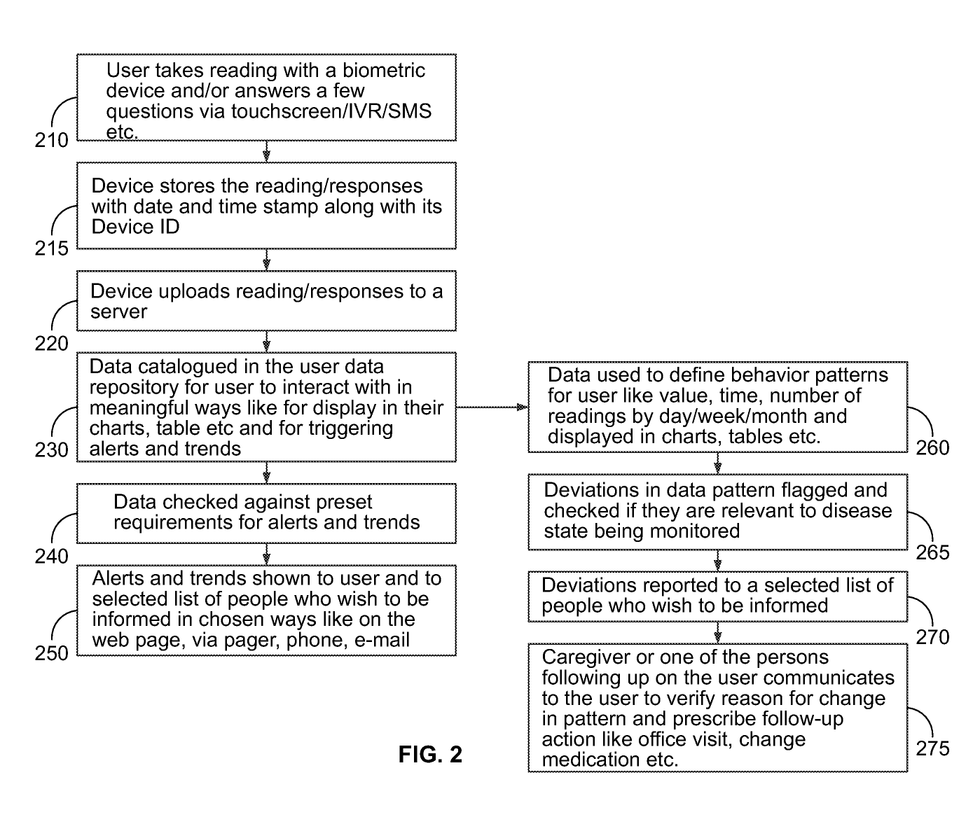

FIG. 2 illustrates a flowchart 200 of the patient health intervention system. First, at step 210, a patient or user takes a reading with a biometric device. Alternatively or additionally, the user may answer one or more questions, and may do so using any of several systems, such as touchscreen, Interactive Voice Response (IVR), or Short Message Service (SMS), for example.

Next, at step 215, the biometric and/or other device may store the reading and/or responses and may associate a date stamp, a time stamp, and a DeviceID with the readings. Next, at step 220, the device uploads the reading/and or responses to a server.

At step 230, once the readings/responses are received by the server, the reading data is catalogued in a user data repository along with other patient specific data like diagnosis, claims history, demographic information. The user may then interact with the data in meaningful ways, such as displaying the data in charts or tables. Additionally, the data may be used to trigger alerts and/or to determine trends or a normal condition.

Then, at step 240, the data that has been received from the patient is checked against preset requirements for alerts and/or trends. The alerts and/or trends at step 240 may be generalized population-wide measurements that may trigger an alert, such as any systolic blood pressure reading over 180, for example.

Next, at step 250, the alerts and/or trends may be shown to the patient and may also be shown to a selected list of other people such as doctors, nurses, or other caregivers, family members, or employers. Additionally, the alerts and/or trends may be transmitted to the desired persons using any of a variety of methodologies, such as making them available on an internet web page or through a pager, phone and/or e-mail.

Additionally, as recited at step 260, the data received from the patient may be used to define or refine a model of the patient's behavior patters, such as the value, time, and number of readings. Additionally, the data may be analyzed in a variety of time periods such as day, week, and/or month. Further, the data may be displayed in charts and tables.

At step 265, the data received from the patient is compared against the model of the patient's normal behavior patterns. Deviations from the normal patterns are flagged and checked to see if they are relevant to the disease state being monitored.

Relevant disease state may be for example heart failure, diabetes, asthma, hypertension, COPD, obesity, Macular degeneration etc. Biometric data measured for heart failure patient would be blood pressure, weight and/or pulse oximeter readings.

At step 270, the deviations may be reported to a selected list of people who wish to be informed, such as the patient, a doctor, nurse or caregiver, and/or a relative or family member.

Finally, at step 275 a caregiver or one of the other persons receiving the data may follow up with the user and attempt to review the reason for the deviation from the normal behavior patents and see if it may be medically relevant. If desirable, a follow-up visit such as an office visit may be scheduled and/or the patient's medication may be changed.

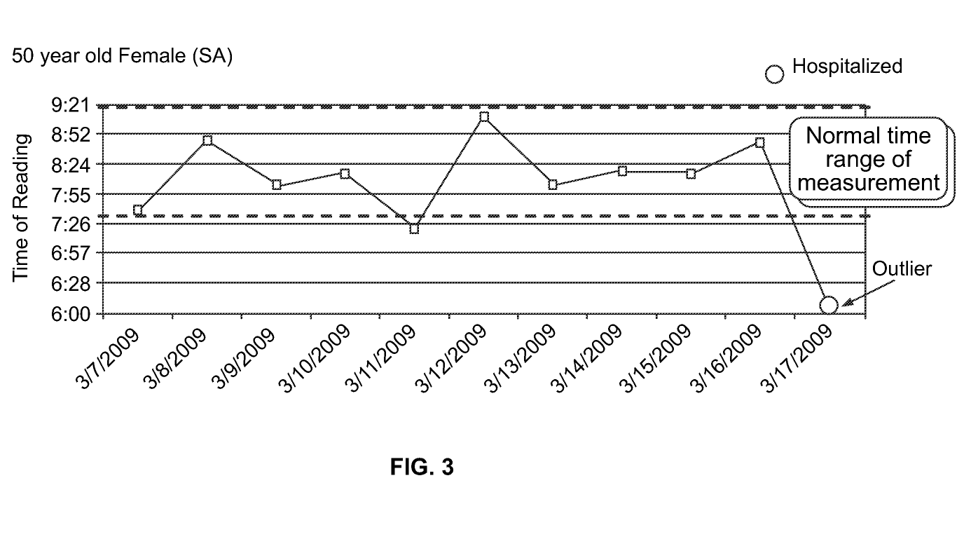

FIG. 3 illustrates an example of the prediction of the onset of a health problem. As shown in FIG. 3, a 50 year old female had medical issues with respect to her blood pressure and had been instructed to measure her blood pressure once a day. Based on her previous readings (including for dates before March 7^{th}) a “normal” range for her time of reading had been determined to be between 7:34 am and 9:18 am. However, on March 17^{th}, the patient took her reading at 6 am—far outside the normal range. In fact, the patient of FIG. 3 ended up being hospitalized that same day and thus the variation in time of reading was a good predictor of the onset of a health problem.

There are several ways to determine the “normal” state for a patient's reading values and times. For example, a moving average, also called rolling average, rolling mean or running average may be employed—and may assist in smoothening small variations. The window for estimating an average may be as small as 3 days or as large as a month or a year to evaluate various trends.

An additional method is to employ Bollinger Bands which include: 1) a middle band being an N-period simple moving average (MA), 2) an upper band at K times an N-period standard deviation above the middle band (MA+Kσ), and 3) a lower band at K times an N-period standard deviation below the middle band (MA−Kσ). In this situation, when a value breaks through the bands, it may be viewed as deviating and trigger a notification.

Similarly, Average True Range (ATR) may be used for trend analysis. The average true range is an N-day exponential moving average of the true range values. A 7-day period may be used for adequate smoothening.

Further, multivariate analysis may provide improved predictability of an adverse event or decomposition. For example, a correlation of diagnosis, previous claims history, age and/or values of readings transmitted and change thereof.

In another embodiment, the system may determine a “health problem likelihood score” based on the amount of deviation from the patient's normal readings and values and compare the health problem likelihood score to a threshold to determine if an action will be taken. For example, consider a patient that has been monitoring her blood pressure twice a day for some weeks. An analysis of her previous readings indicates that she has a moving average of 80 for diastolic blood pressure. Further, her standard deviation for blood pressure readings is +/−2, two standard deviations is +/−6.

Additionally, the patient's moving averages of when she takes her readings are 9:04 am and 5:37 pm, with a standard deviation of +/−14 minutes and +/−26 minutes respectively, and two standard deviations of +/−35 minutes and +/−55 minutes respectively. Additionally, the patient has always taken only two readings a day.

During normal operation, if the patient's reading values and reading times are inside one standard deviation of the averages, then the likelihood that there is a health problem is low.

However, today the patient takes a reading and gets a value of 83—outside of one standard deviation, but inside of two standard deviations. In this case, the system may wait to see if the net reading is also outside of one standard deviation and only indicate an abnormal condition when there are two or more such consecutive readings. Alternatively, the system may immediately indicate an abnormal condition.

Additionally, if the patient takes a reading outside of two standard deviations, the system may immediately indicate an abnormal condition. Alternatively, the system may wait to see if the next reading is also outside one or two standard deviations and only indicate an abnormal condition when there are two or more such consecutive readings.

Similarly, with regard to time of reading, if the time of reading exceeds two standard deviations, then the system may immediately indicate an abnormal condition. If the time of reading is greater than one standard deviation, but less than two, the system may wait and only indicate an abnormal condition if the next two or more readings also exceed one standard deviation. Alternatively, the system may use the alternates described above with regard to the blood pressure value.

Additionally, the system may combine data analysis for both blood pressure reading value and blood pressure time. For example, if the blood pressure reading value and blood pressure time are both more than one standard deviation, but less then two standard deviations away from average, then instead of waiting for another reading, the system may immediately indicate an abnormal condition. That is, although the system would typically wait for further readings if either of the blood pressure reading value and reading time alone were more than one but less than two standard deviations away from average, the fact that both are now deviating causes the system to immediately shift to an abnormal condition.

In addition to the description above, the system may also keep separate records for week days and week ends and separately track the averages and other data. Consequently, the system automatically recognizes whether a user is performing a reading on a week day or a week end and applies the correct information set.

Additionally, there may be an increased likelihood of a problem if the measurement time is during normal sleeping hours. For example, if the system is configured to require multiple abnormal readings before indicating an abnormal condition, then if the patient starts taking readings between midnight and 5 am, then the system may automatically shift to an abnormal condition without the need for an additional reading.

Further, other medical factors may cause a doctor, nurse, or other caregiver to adjust the system's sensitivity. For example, if a patient has been released from the hospital in the last 10 days, then the system may indicate than an abnormal condition has occurred with only a single reading more than one standard deviation, but less than two—even though with a regular patient it would typically require multiple readings outside of one standard deviation. A similar method may be employed if a patient has changed their medicine during a recent time, such as within the last 10 days.

Additionally, the system may be implemented to provide more than one threshold for care. For example, if both blood pressure value and time are more than one standard deviation off, a “check-in” threshold may be reached wherein the nurse, doctor, or other caregiver then checks-in with the patient, for example by phone.

Alternatively, if both blood pressure value and time are more than two standard deviations off, then a “major problem” threshold for increased activity may be reached and more aggressive action may be taken such as 1) sending a nurse or ambulance to the patient, or 2) demanding that the patient immediately visit the doctor's office.

Additionally, although the biometric used in the above examples has been blood pressure, additional biometrics may be employed such as weight, glucose level

Other biometric values that may be used are, for example, Temperature, Blood oxygen, Insulin, Peak expiratory flow, Forced expiratory volume, Prothromin time (PT/INR), C-Reactive Protein, Creatine, Blood gas and electrolytes like Sodium, Potassium, Ionized Calcium, Hematocrit, Chloride, Urea Nitrogen, pH, PO₂, PCO₂, TCO₂, HCO₃, SO₂, Hemoglobin, Visual Acuity etc.

Biometric values and time may be collected using sensor devices in home or in lab. They may be further transferred to the Central server using a hub using a phone line, internet, or cellular networks directly from the measuring sensor. Alternatively, the biometric values and time may be transcribed from the sensor by the patient or someone else and entered into a data entry system to then transfer to the central server. The data entry system may be for example a phone, tablet, PC, touch screen or keyboard device, transferring over the phone line, internet or cellular networks. Alternatively, the transcribed data may be reported via IVR. SMS, email, twitter etc. over the phone line, internet or cellular networks, for example.

Normal values may be in the context of time of day, day of week, day of month, etc. They may also be based on a number of readings say 5 or for a week to develop a baseline. Deviation from normal may be captured by looking at absolute values, rate of change biometric values, change in number of readings for a given period, rate of change in number of readings for a given period, change in time of taking the reading, etc, for example.

Many people who are experiencing or about to experience a health problem start feeling uncomfortable without truly understanding why. Additionally, they may be experiencing vague symptoms, such as feeling bloated, warm, weak etc. When they do, they may check their temperature, blood pressure, weight, peak flow, glucometer etc. more often on a day when they feel uncomfortable. When patients feel uncomfortable, they may also take fewer readings than there is their normal practice. Also, a person who feels uncomfortable may take readings at an earlier or later time than normal. Normal is specific to a person and could be different for other people. Any change in normal behavior could be predictor for an impending change in health condition or an early indication that health has or is changing. Usually such changes are not even captured or identified, and may even be ignored until the health condition has further deteriorated.

In addition to patterns of monitoring their health, there is a natural variation of a person and their vital signs. A person's daily vitals signs may be grouped for example by time of day (morning, noon, evening etc.), or day of week, or months/seasons. For example, asthma attacks happen in September, October, January and March. As such peak flow readings change during the period. There is also variation of weight from summer to winter months. Routines are different during the week impacting blood pressure and glucometer readings for example, on Monday, Friday and weekends. Once these readings are accounted for in a historical record, changes in vital signs can be identified and thus capture changes in health condition. A gain of say 10 points in Systolic or Diastolic blood pressure or 2-3 pounds change in weight could be indicator of deterioration in health condition. For ascites patients, who collect fluid in their abdomen, a change of say 4 lbs could cause them to get admitted. A timely intervention would cause them to consult with their physician and adjust medication as needed.

Regular vital signs from a person may be used in the present system along with other information such as demographics, HRA, pharmaceutical fill/refill data to identify and to predict changes in health condition. Based on history of vital signs, one may identify a trend which predicts that if this trend continues when combined with other information on the patient, patient is likely to decompensate soon. For example, in last 2 months the user has gained 5 lbs and would possibly continue to gain in next months if the trend continues. Trends may be developed on value of reading, time of reading, number of readings in a period and rate of change of the value, time or number of readings. Rate of change in Biometric reading is normally based on population models like 5 pounds weight gain in a week. For a given patient, based on their normal weight, a change of 4 pounds in a week or increase may be an alert condition and will be specific to their “normal”, diagnosis, claims history, demographic factor etc. Similarly, for another patient, a gain of 4 lbs in a 10 days and simultaneous change in blood sugar level of 10 points in the same period may be an alert condition.

In this case a notification is provided to the patient, caregiver, or well wisher. The notified person/system gets in touch with the patient with a timely intervention. The intervention could be, for example, to change medication, increase dosage, add medication, hospitalize etc.

In one embodiment, users are provided devices to report biometric data and/or subjective data as needed. They are asked to report readings as and when needed. The devices and subjective data reporting tools upload data to a central server. The data is then made available to users, caregivers and/or well wishers as needed.

This data when trended also can be used to profile the user. This allows for a baseline to be created on information like when they take readings, how many readings they take in a day, average values of the readings etc.

Deviations form the baseline trigger a notification to the person following up o the user. For example, a caregiver could then be notified by email, IVR, SMS etc to alert them of any deviation.

When checked against known diagnosis for the user, the caregiver could then communicate with the patient to adjust medication or bring them into the office. This could potentially prevent a hospitalization.

While particular elements, embodiments, and applications of the present invention have been shown and described, it is understood that the invention is not limited thereto because modifications may be made by those skilled in the art, particularly in light of the foregoing teaching. It is therefore contemplated by the appended claims to cover such modifications and incorporate those features which come within the spirit and scope of the invention.

1. A method of personalizing a care program for a person and determining an appropriate intervention, the method comprising:
acquiring data over a first period time that characterizes the person's health;
identifying characteristics of the data that was acquired during the first period of time;
comparing subsequent data to characteristics of the data acquired during the first period of time;
identifying a deviation of subsequent data from the characteristics of data acquired during the first period of time; and
determining whether the identified deviation indicates change in health for which an intervention is appropriate.

2. The method of personalizing a care program for a person and providing timely intervention of claim 1, wherein the data acquired during the first period includes timing and frequency of taking of health measurements.

3. The method of personalizing a care program for a person and providing timely intervention of claim 2, wherein the data acquired during the first period includes health measurements.

4. The method of personalizing a care program for a person and providing timely intervention of claim 3, wherein the health measurements include body temperature.

5. The method of personalizing a care program for a person and providing timely intervention of claim 1, wherein the data acquired during the first period includes demographic, genetic history, and pre-conditions.
